(19) United States
(12) Patent Application Publication
(10) Pub. No.: US 2017/0079721 A1
(43) Pub. Date: Mar. 23, 2017

(54) METHOD AND SYSTEM FOR CONSTRUCTING PROSTHESIS FOR DEFECT PART OF TISSUES AND ORGANS

(71) Applicant: Shenzhen Excellent Technology Company Limited, Shenzhen (CN)

(72) Inventors: Xiaowen Zhao, Shenzhen (CN); Dongfeng Zhang, Shenzhen (CN); Wenping Zhao, Shenzhen (CN); Junhua Cai, Shenzhen (CN)

(21) Appl. No.: 15/145,919

(22) Filed: May 4, 2016

(30) Foreign Application Priority Data
Sep. 23, 2015 (CN) 201510610364.6

(51) Int. Cl. A61B 34/10; G06F 17/50; B33Y 50/02; B33Y 80/00; A61F 2/30; A61F 2/36

(52) U.S. Cl. CPC A61B 34/10; A61F 2/30942; A61F 2/36; B33Y 50/02; B33Y 80/00; G06F 17/5009; A61B 2034/102; A61B 2034/108; A61B 2034/105; A61F 2002/30985; A61F 2002/30948

(57) ABSTRACT

A method and a system for constructing prosthesis for defect part of tissues and organs are provided. The method includes the steps of obtaining a tissue defect site of a patient; collecting original image data of the tissue defect site of the patient, restoring an original three-dimensional image of the tissue defect site and a three-dimensional image of a defect part corresponding to the defect site, and generating and storing a three-dimensional model for mending the defect part; performing simulated mending on the basis of the three-dimensional model for mending the defect part and a tissue defect model; obtaining a three-dimensional model for mending the defect part, printing the three-dimensional model for mending the defect part and generating a physical model of the prosthesis.

### TECHNICAL FIELD

The present invention refers to the technical field of tissue engineering repair, particularly to a method and system for constructing prosthesis for defect part of tissues and organs.

### BACKGROUND

A regenerative-type biological patch is extracted from natural organisms through the use of a series of technologies such as advanced cross-linking fixation, multifold antigen-removing, etc. It can be comprehended as “cytoskeleton”. According to the principles of regenerative medicine, molecular biology, and immunology, the regenerative-type biological patch can play a good stent role, and fill lost tissue in a defect part after being implanted in a human body. Moreover, new tissue can gradually grow up at an original position with the body's self-replenishing function induced by the material to replace the biological material and complete the regeneration process of tissues and organs. At an early stage, it is mainly used in neurosurgery, endomeninx repair, repair after resection of esophageal cancer, skin and pleura repair after incision of Siamese twins, skull repair, gynecology, andrology, lung cancer, tumor, etc., as well as the rescue of patients who are burned over a large surface area, and now the use in the field of cosmetic surgery has started to be explored.

Tissue defect repair is a difficult medical problem in the whole world, and only the patch can be used to cover the surface of a defect at present. However, the patch cannot satisfy a defect part with complex physiological structure. It cannot fill an irregular part and be used to individually match a defect part with a large volume and a large area. Moreover, the patch is a single-typed product and cannot satisfy all kinds of tissue defects and individuality cannot be achieved.

Therefore, the prior art should be improved and enhanced.

### BRIEF SUMMARY OF THE INVENTION

In view of the shortcomings in the prior art, the object of the present invention is to provide a method and system for constructing prosthesis for defect part of tissues and organs in order to solve drawbacks in the prior art that the medical patch only can be used to cover a surface of a defect tissue but cannot fill an irregular defect part and match a defect part with a large volume and a large area.

The technical solution of the present invention is as follows:

A method for constructing prosthesis for defect part of tissues and organs includes the steps:

A. pre-storing medical image data of tissues and organs of a patient's body obtained by medical imaging technologies, and physiological structure data and anatomical structure data of tissues and organs of the patient;

B. obtaining actual image data of currently detected tissues of the patient and comparing with the pre-stored medical image data to obtain a tissue defect site of the patient;

C. converting the pre-stored original medical image data into a three-dimensional image, collecting original image data of the tissue defect site of the patient, restoring an original three-dimensional image of the tissue defect site and a three-dimensional image of a defect part corresponding to the defect site, and generating and storing a three-dimensional model for mending the defect part;

D. performing simulated mending on the basis of the three-dimensional model for mending the defect part and a tissue defect model until the characteristics of the three-dimensional structure after simulated mending and the whole pre-defect tissue structure are completely matched; and

E. obtaining and storing the three-dimensional model for mending the defect part after the defect part is removed, printing the three-dimensional model for mending the defect part with a multi-dimensional printer and generating a physical model of the prosthesis.

The method for constructing prosthesis for defect part of tissues and organs further includes the following step after step E:

F. performing a surface treatment to the physical model of the prosthesis in accordance with the requirements of mending the defect tissue part.

The method for constructing prosthesis for defect part of tissues and organs further includes the following steps after step F:

F1. obtaining a three-dimensional image from scanning the physical model of the prosthesis in a three-dimensional mode, comparing with a pre-stored defect prototype or matching the physical model of the prosthesis with the defect model made by three-dimensional printing, and determining whether the prosthesis made by three-dimensional printing meets the preset design requirements;

F2. determining the prosthesis is a qualified product if the design requirements are met; and

F3. reprinting the physical model of the prosthesis if the design requirements are not met.

The method for constructing prosthesis for defect part of tissues and organs, wherein the step D particularly includes:

D1. obtaining a three-dimensional structure after simulated mending on the basis of the simulated mending between the three-dimensional model for mending the defect part and the tissue defect part; and

D2. performing superimposition repeatedly during the simulated mending process until the characteristics of the three-dimensional structure after simulated mending and the whole pre-defect tissue structure are completely matched.

Any one of the above methods for constructing prosthesis for defect part of tissues and organs, wherein the medical imaging technologies includes: X-ray imaging, ultrasonic imaging, electronic computer tomography (CT) imaging, magnetic resonance imaging (MRI) and positron emission tomography-computed tomography (PET-CT).

A system for constructing prosthesis for defect part of tissues and organs, wherein the system includes:

a pre-storage module for pre-storing medical image data of tissues and organs of a patient's body obtained by medical imaging technologies, and physiological structure data and anatomical structure data of the tissues and organs;

a tissue defect site obtaining module for obtaining actual image data of currently detected tissues of the patient and comparing with the pre-stored medical image data to obtain a tissue defect site of the patient;

an image processing and storing module for converting the pre-stored original medical image data into a three-dimensional image, collecting original image data of the tissue defect site of the patient, restoring an original three-dimensional image of the tissue defect site and a three-dimensional image of a defect part corresponding to the defect site, and generating and storing a three-dimensional model for mending the defect part;

a simulated mending module for performing simulated mending on the basis of the three-dimensional model for mending the defect part and a tissue defect model until the characteristics of the three-dimensional structure after simulated mending and the whole pre-defect tissue structure are completely matched;

a prosthesis model printing module for obtaining and storing the three-dimensional model for mending the defect part after the defect part is removed, printing the three-dimensional model for mending the defect part with a multi-dimensional printer and generating a physical model of the prosthesis.

The system for constructing prosthesis for defect part of tissues and organs further includes:

a surface treatment module for performing a surface treatment to the physical model of the prosthesis in accordance with the requirements of mending the defect tissue part.

The system for constructing prosthesis for defect part of tissues and organs, wherein the system further includes:

a comparison module for obtaining a three-dimensional image from scanning the physical model of the prosthesis in a three-dimensional mode, comparing with a pre-stored defect prototype or matching the physical model of the prosthesis with the defect model made by three-dimensional printing, and determining whether the prosthesis made by three-dimensional printing meets the preset design requirements;

a determining module for determining a qualified product if the design requirements are met; and

a control module for reprinting the physical model of the prosthesis if the design requirements are not met.

The system for constructing prosthesis for defect part of tissues and organs, wherein the simulated mending module particularly includes:

a mending unit for obtaining a three-dimensional structure after simulated mending on the basis of the simulated mending between the three-dimensional model for mending the defect part and the tissue defect part; and

a matching and superimposing unit for performing superimposition repeatedly during the simulated mending process until the characteristics of the three-dimensional structure after simulated mending and the whole pre-defect tissue structure are completely matched.

Any one of the above systems for constructing prosthesis for defect part of tissues and organs, wherein the medical imaging technologies includes: X-ray imaging, ultrasonic imaging, electronic computer tomography (CT) imaging, magnetic resonance imaging (MRI) and positron emission tomography-computed tomography (PET-CT).

A method and system for constructing prosthesis for defect part of tissues and organs are provided. The method includes the following steps: obtaining tissue defect site of a patient; collecting original image data of the tissue defect site of the patient, restoring an original three-dimensional image of the tissue defect site and a three-dimensional image of a defect part corresponding to the defect site, and generating and storing a three-dimensional model for mending the defect part; performing simulated mending on the basis of the three-dimensional model for mending the defect part and a tissue defect model; and obtaining a three-dimensional model for mending the defect part, printing the three-dimensional model for mending the defect part and generating a physical model of the prosthesis. A defect mending part is planned and designed on the basis of the characteristics of the defect tissue structure to make it match with the original physiological structure, and then the defect mending part is printed in a 3D-printing mode for filling and repairing the defect part. Thus the whole tissue structure is fully reproduced with pre-defect intact effect, and any complex defect part with a large volume and a large area can be individually repaired.

### BRIEF DESCRIPTION OF THE DRAWING

FIG. 1 is a flow chart of a method for constructing prosthesis for defect part of tissues and organs according to one embodiment of the present invention.

FIG. 2 is a schematic view of calculating and displaying the tissue defect characteristics in a method for constructing prosthesis for defect part of tissues and organs according to one embodiment of the present invention.

FIG. 3 is a schematic view of identifying and comparing a tissue defect site in a method for constructing prosthesis for defect part of tissues and organs according to one embodiment of the present invention.

FIG. 4 is a schematic view of restoring and matching a defect part in a method for constructing prosthesis for defect part of tissues and organs according to one embodiment of the present invention.

FIG. 5 is a schematic view of generating prosthesis in a method for constructing prosthesis for defect part of tissues and organs according to one embodiment of the present invention.

FIG. 6 is a functional block diagram of a system for constructing prosthesis for defect part of tissues and organs according to one embodiment of the present invention.

### DETAILED DESCRIPTION

In order to make the purpose, technical solution and effects of the present invention clearer and more explicit, The present invention is further illustrated in detail below. However, it should be understood that the particular embodiments described here are for illustrative purposes only.

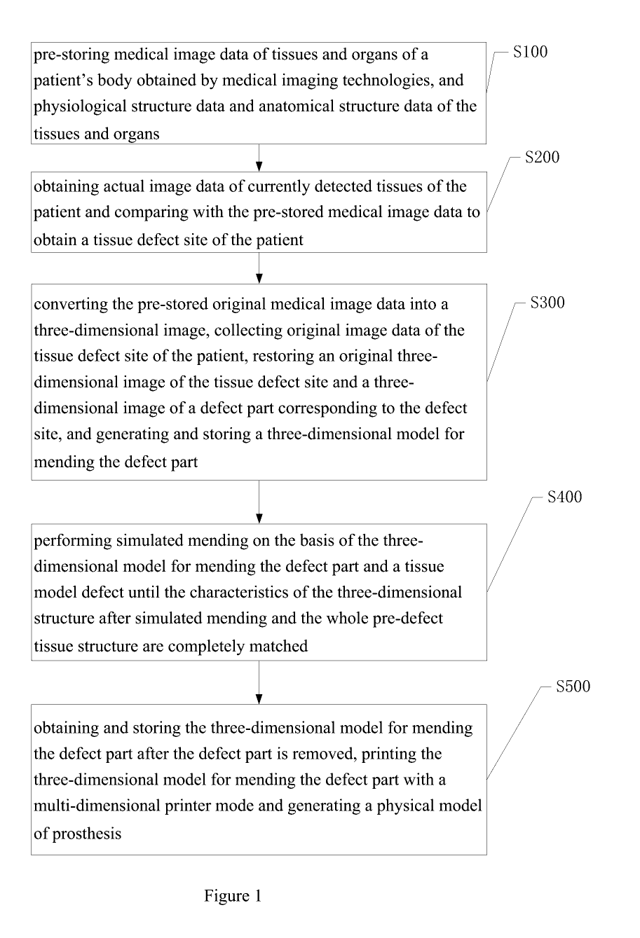

In accordance with one embodiment, a method for constructing prosthesis for defect part of tissues and organs, as shown in FIG. 1, includes:

Step S100, pre-storing medical image data of tissues and organs of a patient's body obtained by medical imaging technologies, and physiological structure data and anatomical structure data of the tissues and organs.

In practice, the medical image data, physiological structure data and anatomical structure data of human body's respective tissues and organs are obtained by medical imaging technologies when the patient is in normal condition. The medical imaging technologies includes: X-ray imaging, ultrasonic imaging, CT (Computed Tomography), MM (Magnetic Resonance Imaging) and PET-CT (Positron Emission Computed Tomography). Specifically, the physiological tissue structures and the anatomical structures are analyzed by obtaining medical images and original data of X-ray, ultrasonic, CT, MM, PET-CT, etc.

Step S200, obtaining actual image data of currently detected tissues of the patient and comparing with the pre-stored medical image data to obtain a tissue defect site of the patient.

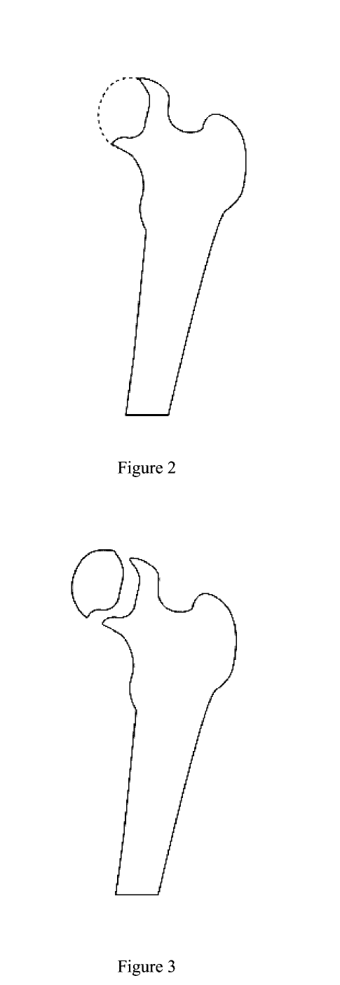

In practice, as shown in FIG. 2, a defect part is verified and the characteristics thereof are calculated. Images are converted to three-dimensional graphics and then displayed in a two-dimensional mode. Original imaging CT data of the patient's lesion is collected and imported into a computer, and a three-dimensional image of the tissue and an image of the specific defect part are obtained by the use of precise restoration.

Step S300, converting the pre-stored original medical image data into a three-dimensional image, collecting original image data of the tissue defect site of the patient, restoring an original three-dimensional image of the tissue defect site and a three-dimensional image of a defect part corresponding to the defect site, and generating and storing a three-dimensional model for mending the defect part.

In practice, as shown in FIG. 3, the obtained original data is recognized by a machine and feature comparison is performed for many times to rebuild a three-dimensional model. The above three-dimensional graphics are analyzed and segmented, and defect part planning is performed to obtain the inverse data that corresponds precisely to residual parts. Subsequently, the three-dimensional models of the defect part and the residual parts are designed. The three-dimensional models are stored or converted to files with the file format of stl, stp, obj, max, 3ds, ma, vtk, igs, etc.

Step S400, performing simulated mending on the basis of the three-dimensional model for mending the defect part and a tissue defect model until the characteristics of the three-dimensional structure after simulated mending and the whole pre-defect tissue structure are completely matched.

In practice, the step S400 particularly includes:

Step S401, obtaining a three-dimensional structure after simulated mending on the basis of the simulated mending between the three-dimensional model for mending the defect part and the tissue defect part; and

Step S402, performing superimposition repeatedly during the simulated mending process until the characteristics of the three-dimensional structure after simulated mending and the whole pre-defect tissue structure are completely matched.

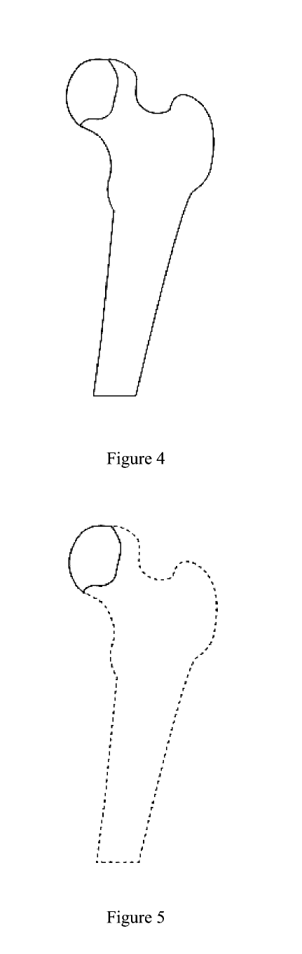

In practice, as shown in FIG. 4, simulated mending is performed on the basis of a defect part of a 3D printing model and a model for residual parts. Pre-defect original appearance is simulated on the basis of the characteristics of the defect part, and the original features of the whole pre-defect tissue structure and the three-dimensional structure are obtained to make the complementary and mended structure completely match and mend the defect part. Pre-defect original appearance is simulated on the basis of individualized tissue structure data, data or structure of physiological structure of an organ, defect tissue or organ contour of a three-dimensional structural part of tissue, and structural parts with good symmetry. Superimposing is performed repeatedly during the simulated match process to make the defect part together with the mended and filled part meet the requirements, and thus a defect mending three-dimensional structure is obtained.

Step S500, obtaining and storing the three-dimensional model for mending the defect part after the defect part is removed, printing the three-dimensional model for mending the defect part with a multi-dimensional printer and generating a physical model of prosthesis.

In practice, as shown in FIG. 5, the defect part is removed and defect mending part is left behind, which is saved as a file with the file format of stl, stp, obj, max, 3ds, ma, vtk, igs, etc., so as to be used for transmission, storage, browse, examination, modification and manufacture. The defect mending part is made by 3D printing and a physical model is realized quickly. The defect mending part is printed and manufactured as a 1:1 physical model by 3D printer or other multi-dimensional printer such as 4D printer, 5D printer, etc.

Furthermore, the method further includes the following step after Step S500:

Step S600, performing a surface treatment to the physical model of the prosthesis in accordance with the requirements of mending the defect tissue part. Specifically, proper surface treatment is performed to the physical model in accordance with the effect of 3D printing and the requirement of use is achieved, making better matching and melding between the physical model and the defect tissues.

In practice, the method further includes the following steps after Step S600:

Step S701, obtaining a three-dimensional image from scanning the physical model of the prosthesis in a three-dimensional mode, comparing with a pre-stored defect prototype or matching the physical model of the prosthesis with the defect model made by three-dimensional printing, and determining whether the prosthesis made by three-dimensional printing meets the preset design requirements;

Step S702, determining the prosthesis is a qualified product if the design requirements are met; and

Step S703, reprinting the physical model of the prosthesis if the design requirements are not met.

In practice, 1) the defect mending model made by 3D printing is scanned in a 3D mode, the resulted three-dimensional graphics are measured and compared with a design prototype in a computer to examine whether it meets the preset design requirements. 2) a matching experiment is performed to the defect mending model made by 3D printing and the defect model made by 3D printing to examine whether the preset requirements are met. The prosthesis is determined to be a qualified product if the design requirements are met; and the physical model of the prosthesis is printed again if the design requirements are not met, and thus the manufactured physical model of the prosthesis is guaranteed to meet practical needs and the adverse effects to the human body are avoided.

It can be known from the above method embodiments that a method for constructing prosthesis for defect part of tissues and organs is provided. A defect mending part is planned and designed on the basis of the characteristics of the defect tissue structure to make it match with the original physiological structure, and then the defect mending part is printed in a 3D-printing mode for filling and repairing the defect part. Thus the whole tissue structure is fully reproduced with pre-defect intact effect, and any complex defect part with a large volume and a large area can be individually repaired.

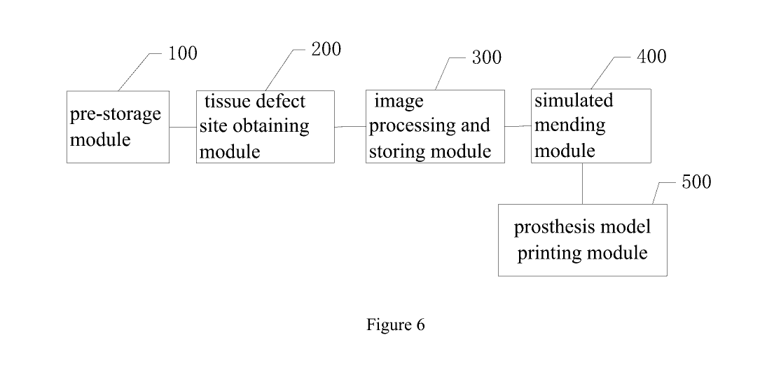

A functional block diagram of one embodiment of a system for constructing prosthesis for defect part of tissues and organs is provided by the present invention on the basis of the above method embodiments, as shown in FIG. 6, and the system includes:

a pre-storage module 100 for pre-storing medical image data of tissues and organs of a patient's body obtained by medical imaging technologies and physiological structure data and anatomical structure data of the tissues and organs, as mentioned above in details;

a tissue defect site obtaining module 200 for obtaining actual image data of currently detected tissues of the patient and comparing with the pre-stored medical image data to obtain a tissue defect site of the patient, which is mentioned above in details;

an image processing and storing module 300 for converting the pre-stored original medical image data into a three-dimensional image, collecting original image data of the tissue defect site of the patient, restoring an original three-dimensional image of the tissue defect site and a three-dimensional image of a defect part corresponding to the defect site, and generating and storing a three-dimensional model for mending the defect part; as mentioned above in details;

a simulated mending module 400 for performing simulated mending on the basis of a three-dimensional model for mending the defect part and a tissue defect model until the characteristics of the three-dimensional structure after simulated mending and the whole pre-defect tissue structure are completely matched, as mentioned above in details;

a prosthesis model printing module 500 for obtaining and storing the three-dimensional model for mending the defect part after the defect part is removed, printing the three-dimensional model for mending the defect part with a multi-dimensional printer and generating a physical model of the prosthesis, as mentioned above in details.

The system for constructing prosthesis for defect part of tissues and organs further includes:

a surface treatment module for performing a surface treatment to the physical model of the prosthesis in accordance with the requirements of mending the defect tissue part; as mentioned above in details.

The system for constructing prosthesis for defect part of tissues and organs further includes:

a comparison module for obtaining a three-dimensional image from scanning the physical model of the prosthesis in a three-dimensional mode, comparing with a pre-stored defect prototype or matching the physical model of the prosthesis with the defect model made by three-dimensional printing, and determining whether the prosthesis made by three-dimensional printing meets the preset design requirements, as mentioned above in details;

a determining module for determining a qualified product if the design requirements are met, as mentioned above in details;

a control module for re-printing the physical model of the prosthesis if the design requirements are not met, as mentioned above in details.

The system for constructing prosthesis for defect part of tissues and organs, wherein the simulated mending module particularly includes:

a mending unit for obtaining a three-dimensional structure after simulated mending on the basis of the simulated mending between the three-dimensional model for mending the defect part and the tissue defect part, as mentioned above in details;

a matching and superimposing unit for performing superimposition repeatedly during the simulated mending process until the characteristics of the three-dimensional structure after simulated mending and the whole pre-defect tissue structure are completely matched, as mentioned above in details.

Any one of the above system for constructing prosthesis for defect part of tissues and organs, wherein the medical imaging technologies includes: X-ray imaging, ultrasonic imaging, electronic computer tomography (CT) imaging, magnetic resonance imaging (MRI) and positron emission tomography-computed tomography (PET-CT), as mentioned above in details.

In conclusion, a method and system for constructing prosthesis for defect part of tissues and organs are provided. The method includes the following steps: obtaining tissue defect site of a patient; collecting original image data of the tissue defect site of the patient, restoring an original three-dimensional image of the tissue defect site and a three-dimensional image of a defect part corresponding to the defect site, and generating and storing a three-dimensional model for mending the defect part; performing simulated mending on the basis of a three-dimensional model for mending the defect part and a tissue defect model; and obtaining a three-dimensional model for mending the defect part, printing the three-dimensional model for mending the defect part and generating a physical model of the prosthesis. A defect mending part is planned and designed on the basis of the characteristics of the defect tissue structure to make it match with the original physiological structure, and then the defect mending part is printed in a 3D-printing mode for filling and repairing the defect part. Thus the whole tissue structure is fully reproduced with pre-defect intact effect, and any complex defect part with a large volume and a large area can be individually repaired.

It should be understood by those skilled in the art that the application of the present invention is not limited to the above examples, improvements and changes can be made on the basis of the above description, and all these improvements and changes should be included within the scope of the appended claims of the present invention.

1. A method for constructing prosthesis for defect part of tissues and organs, the method comprising:
A. pre-storing medical image data, physiological structure data, and anatomical structure data of tissues and organs of a patient that are obtained by medical imaging technologies;
B. obtaining actual image data of the tissues and organs of the patient and comparing the actual image data with the pre-stored medical image data to obtain a tissue defect site of the patient;
C. converting the pre-stored medical image data into a three-dimensional image, collecting original image data of the tissue defect site position of the patient, restoring an original three-dimensional image of the tissue defect site and a three-dimensional image of a defect part corresponding to the tissue defect site, and generating and storing a three-dimensional model for mending the defect part;
D. repeating simulated mending based on the three-dimensional model for mending the defect part and a tissue defect model until characteristics of a three-dimensional structure after simulated mending and a whole pre-defect tissue structure are completely matched; and
E. obtaining and storing the three-dimensional model for mending the defect part after the defect part is removed, printing the three-dimensional model for mending the defect part with a multi-dimensional printer and generating a physical model of the prosthesis.

2. The method for constructing prosthesis for defect part of tissues and organs according to claim 1, wherein the method further comprises the following step after step E:
F. performing a surface treatment to the physical model of the prosthesis for mending the defect part.

3. The method for constructing prosthesis for defect part of tissues and organs according to claim 1, wherein the method further comprises the following steps after step F:
F1. obtaining a three-dimensional image of the prosthesis by scanning the physical model of the prosthesis in a three-dimensional mode, comparing the three-dimensional image of the prosthesis with a pre-stored defect prototype or matching the physical model of the prosthesis with a defect model made by three-dimensional printing, and determining whether the prosthesis meets pre-set design requirements;
F2. determining the prosthesis is a qualified product if the pre-set design requirements are met; and
F3. reprinting the physical model of the prosthesis if the pre-set design requirements are not met.

4. The method for constructing prosthesis for defect part of tissues and organs according to claim 3, wherein the step D further comprises:
D1. obtaining a three-dimensional structure based on the simulated mending that is based on the three-dimensional model for mending the defect part and the defect part; and
D2. performing superimposition repeatedly during the simulated mending until the characteristics of the three-dimensional structure after simulated mending and the whole pre-defect tissue structure are completely matched.

5. The method for constructing prosthesis for defect part of tissues and organs according to claim 1, wherein the medical imaging technologies comprise: X-ray imaging, ultrasonic imaging, electronic computer tomography (CT) imaging, magnetic resonance imaging (MRI), and positron emission tomography-computed tomography (PET-CT).

6. A system for constructing prosthesis for defect part of tissues and organs, the system comprising:
a pre-storage module for pre-storing medical image data of tissues and organs of a patient obtained by medical imaging technologies, and physiological structure data and anatomical structure data of the tissues and organs;
a tissue defect site obtaining module for obtaining actual image data of the tissues and organs and comparing the actual image data with the pre-stored medical image data to obtain a tissue defect site of the patient;
an image processing and storing module for converting the pre-stored medical image data into a three-dimensional image, collecting original image data of the tissue defect site of the patient, restoring an original three-dimensional image of the tissue defect site and a three-dimensional image of a defect part corresponding to the tissue defect site, and generating and storing a three-dimensional model for mending the defect part;
a simulated mending module for performing simulated mending based on the three-dimensional model for mending the defect part and a tissue defect model until the characteristics of a three-dimensional structure after simulated mending and a whole pre-defect tissue structure are completely matched; and
a prosthesis model printing module for obtaining and storing the three-dimensional model for mending the defect part after the defect part is removed, printing the three-dimensional model for mending the defect part with a multi-dimensional printer and generating a physical model of the prosthesis.

7. The system for constructing prosthesis for defect part of tissues and organs according to claim 6, wherein the system further comprises:
a surface treatment module for performing a surface treatment to the physical model of the prosthesis for mending the defect part.

8. The system for constructing prosthesis for defect part of tissues and organs according to claim 7, wherein the system further comprises:
a comparison module for obtaining a three-dimensional image of the prosthesis by scanning the physical model of the prosthesis in a three-dimensional mode, comparing the three-dimensional image of the prosthesis with a pre-stored defect prototype or matching the physical model of the prosthesis with a defect model made by three-dimensional printing, and determining whether the prosthesis meets preset design requirements;
a determining module for determining the prosthesis is a qualified product if the preset design requirements are met; and
a control module for reprinting the physical model of the prosthesis if the preset design requirements are not met.

9. The system for constructing prosthesis for defect part of tissues and organs according to claim 8, wherein the simulated mending module further comprises:
a mending unit for obtaining a three-dimensional structure after simulated mending based on the simulated mending that is based on the three-dimensional model for mending the defect part and the tissue defect part; and
a matching and superimposing unit for performing superimposition repeatedly during the simulated mending until the characteristics of the three-dimensional structure after simulated mending and the whole pre-defect tissue structure are completely matched.

10. The system for constructing prosthesis for defect part of tissues and organs according to claim 6, wherein the medical imaging technologies comprise: X-ray imaging, ultrasonic imaging, electronic computer tomography (CT) imaging, magnetic resonance imaging (MM), and positron emission tomography-computed tomography (PET-CT).

11. The method for constructing prosthesis for defect part of tissues and organs according to claim 2, wherein the medical imaging technologies comprise: X-ray imaging, ultrasonic imaging, electronic computer tomography (CT) imaging, magnetic resonance imaging (MRI), and positron emission tomography-computed tomography (PET-CT).

12. The method for constructing prosthesis for defect part of tissues and organs according to claim 3, wherein the medical imaging technologies comprise: X-ray imaging, ultrasonic imaging, electronic computer tomography (CT) imaging, magnetic resonance imaging (MRI), and positron emission tomography-computed tomography (PET-CT).

13. The method for constructing prosthesis for defect part of tissues and organs according to claim 4, wherein the medical imaging technologies comprise: X-ray imaging, ultrasonic imaging, electronic computer tomography (CT) imaging, magnetic resonance imaging (MRI), and positron emission tomography-computed tomography (PET-CT).

14. The system for constructing prosthesis for defect part of tissues and organs according to claim 7, wherein the medical imaging technologies comprise: X-ray imaging, ultrasonic imaging, electronic computer tomography (CT) imaging, magnetic resonance imaging (MM), and positron emission tomography-computed tomography (PET-CT).

15. The system for constructing prosthesis for defect part of tissues and organs according to claim 8, wherein the medical imaging technologies comprise: X-ray imaging, ultrasonic imaging, electronic computer tomography (CT) imaging, magnetic resonance imaging (MM), and positron emission tomography-computed tomography (PET-CT).

16. The system for constructing prosthesis for defect part of tissues and organs according to claim 9, wherein the medical imaging technologies comprise: X-ray imaging, ultrasonic imaging, electronic computer tomography (CT) imaging, magnetic resonance imaging (MM), and positron emission tomography-computed tomography (PET-CT).
